(19) United States
(12) Patent Application Publication
(10) Pub. No.: US 2022/0241359 A1
(43) Pub. Date: Aug. 4, 2022

(54) ATTENUATED YELLOW FEVER VIRUS AND USES THEREOF FOR THE TREATMENT OF CANCER

(71) Applicant: CODAGENIX INC., Farmingdale, NY (US)

(72) Inventors: John Robert Coleman, Blauvelt, NY (US); Steffen Mueller, Great Neck, NY (US); Charles Stauft, Wheatley Heights, NY (US); Ying Wang, South Setauket, NY (US)

(73) Assignee: CODAGENIX INC., Farmingdale, NY (US)

(21) Appl. No.: 17/610,026

(22) PCT Filed: May 14, 2020
(86) PCT No.: PCT/US2020/032901

§ 371 Date: Nov. 9, 2021

Related U.S. Application Data
(60) Provisional application No. 62/848,443, filed on May 15, 2019.

(51) Int. Cl. A61K 35/768; A61K 45/06; A61K 39/12; C12N 7/00

(52) U.S. Cl. CPC A61K 35/768; A61K 45/06; A61K 2039/5254; C12N 7/00; A61K 39/12

(57) ABSTRACT

The present invention is the use of designed recombinant viruses for the treatment of various forms of malignant tumors using attenuated Yellow Fever virus. The method of the present invention is particularly useful for the treatment of malignant tumors in various organs, such as: breast, skin, colon, bronchial passage, epithelial lining of the gastrointestinal, upper respiratory and genito-urinary tracts, liver, prostate and the brain. Astounding remissions in experimental animals have been demonstrated for the treatment of treatment of breast cancer and melanoma.

### CROSS-REFERENCE TO RELATED APPLICATIONS

This application includes a claim of priority under 35 U.S.C. § 119(e) to U.S. provisional patent application No. 62/848,443, filed May 15, 2019, the entirety of which is hereby incorporated by reference.

### FIELD OF INVENTION

The present invention relates to methods of using Yellow Fever virus vaccine strain, modified versions of Yellow Fever virus vaccine strain and modified versions of the Yellow Fever virus to induce oncolytic effects on malignant tumors and to treat malignant tumors.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### Synthetic Virology

Rapid improvements in DNA synthesis technology promise to revolutionize traditional methods employed in virology. One of the approaches traditionally used to eliminate the functions of different regions of the viral genome makes extensive but laborious use of site-directed mutagenesis to explore the impact of small sequence variations in the genomes of virus strains. However, viral genomes, especially of RNA viruses, are relatively short, often less than 10,000 bases long, making them amenable to whole genome synthesis using currently available technology. Recently developed microfluidic chip-based technologies can perform de novo synthesis of new genomes designed to specification for only a few hundred dollars each. This permits the generation of entirely novel coding sequences or the modulation of existing sequences to a degree practically impossible with traditional cloning methods. Such freedom of design provides tremendous power to perform large-scale redesign of DNA/RNA coding sequences to: (1) study the impact of changes in parameters such as codon bias, codon-pair bias, and RNA secondary structure on viral translation and replication efficiency; (2) perform efficient full genome scans for unknown regulatory elements and other signals necessary for successful viral reproduction; (3) develop new biotechnologies for genetic engineering of viral strains and design of anti-viral vaccines; (4) synthesize modified viruses for use in oncolytic therapy.

### De Novo Synthesis of Viral Genomes

Computer-based algorithms are used to design and synthesize viral genomes de novo. These synthesized genomes, exemplified by the synthesis of Yellow Fever virus 17D described herein, can be used to treat cancer.

It has been known that malignant tumors result from the uncontrolled growth of cells in an organ. The tumors grow to an extent where normal organ function may be critically impaired by tumor invasion, replacement of functioning tissue, competition for essential resources and, frequently, metastatic spread to secondary sites. Malignant cancer is the second leading cause of mortality in the United States.

Prior art methods for treating malignant tumors include surgical resection, radiation and/or chemotherapy. However, numerous malignancies respond poorly to all traditionally available treatment options and there are serious adverse side effects to the known and practiced methods. There has been much advancement to reduce the severity of the side effects while increasing the efficiency of commonly practiced treatment regimens. However, many problems remain, and there is a need to search for alternative modalities of treatment.

In recent years, there have been proposals to use viruses for the treatment of cancer: (1) as gene delivery vehicles; (2) as direct oncolytic agents by using viruses that have been genetically modified to lose their pathogenic features; or (3) as agents to selectively damage malignant cells using viruses which have been genetic engineered for this purpose.

Examples for the use of viruses against malignant gliomas include the following. Herpes Simplex Virus dlsptk (HSVdlsptk), is a thymidine kinase (TK)-negative mutant of HSV. This virus is attenuated for neurovirulence because of a 360-base-pair deletion in the TK gene, the product of which is necessary for normal viral replication. It has been found that HSVdlsptk retains propagation potential in rapidly dividing malignant cells, causing cell lysis and death. Unfortunately, all defective herpes viruses with attenuated neuropathogenicity have been linked with serious symptoms of encephalitis in experimental animals. For example, in mice infected intracerebrally with HSVdlsptk, the LD₅₀^{lc} (intracranial administration) is 10⁶ pfu, a rather low dose. This limits the use of this mutant HSV. Other mutants of HSV have been proposed and tested. Nevertheless, death from viral encephalitis remains a problem.

Another proposal was to use retroviruses engineered to contain the HSV tk gene to express thymidine kinase which causes in vivo phosphorylation of nucleoside analogs, such as gancyclovir or acyclovir, blocking the replication of DNA and selectively killing the dividing cell. Izquierdo, M., et al., Gene Therapy, 2:66-69 (1995) reported the use of Moloney Murine Leukemia Virus (MoMLV) engineered with an insertion of the HSV tk gene with its own promoter. Follow-up of patients with glioblastomas that were treated with intraneoplastic inoculations of therapeutic retroviruses by MRI revealed shrinkage of tumors with no apparent short-term side effects. However, the experimental therapy had no effect on short-term or long-term survival of affected patients. Retroviral therapy is typically associated with the danger of serious long-term side effects (e.g., insertional mutagenesis).

Similar systems have been developed to target malignancies of the upper airways, tumors that originate within the tissue naturally susceptible to adenovirus infection and that are easily accessible. However, Glioblastoma multiforme, highly malignant tumors composed of widely heterogeneous cell types (hence the denomination multiforme) are characterized by exceedingly variable genotypes and are unlikely to respond to oncolytic virus systems directed against homogeneous tumors with uniform genetic abnormalities.

The effects of our virus modification can be confirmed in ways that are known to one of ordinary skill in the art. Non-limiting examples induce plaque assays, growth measurements, reverse genetics of RNA viruses, and reduced lethality in test animals. The instant application demonstrates that the modified viruses are capable of inducing protective immune responses in a host as well as inducing an anti-tumor response in the host.

### SUMMARY OF THE INVENTION

The following embodiments and aspects thereof are described and illustrated in conjunction with compositions and methods which are meant to be exemplary and illustrative, not limiting in scope.

It is an objective of the present invention to develop attenuated Yellow Fever Virus (YFV) for the treatment of various types of cancers as further described herein. In various embodiments, the attenuated YFV is Yellow Fever Virus strain 17D vaccine (YFV 17D). In various embodiments, the YFV 17D is synthetic YFV 17D.

It is a further objective of the present invention to develop attenuated Yellow Fever virus (e.g., synthetic YFV 17D) for the treatment for various types of cancer that can be used in combination with anti-PDL-1 antibody therapeutics or other immune-oncology therapies.

It is a further objective of the present invention to treat cancer cells by infecting them with attenuated Yellow Fever virus (e.g., synthetic YFV 17D) to cause cancer cell lysis and death.

It is a further objective of the present invention to treat cancer cells by infecting them with attenuated Yellow Fever virus (e.g., synthetic YFV 17D) and thereby elicit an anti-tumor immune response.

It is a further objective of the present invention to treat cancer cells by infecting them with attenuated Yellow Fever virus (e.g., synthetic YFV 17D) and thereby elicit an anti-tumor immune response by increasing or decreasing the expression of anti-tumor immune proteins such as PD-1, CTLA-4, IDO1, TIM3, lag-3.

It is a further objective of the present invention to treat cancer cells by infecting them with attenuated Yellow Fever virus (e.g., synthetic YFV 17D) and thereby elicit an innate immune response in the tumor.

It is a further objective of the present invention to treat cancer cells by infecting them with attenuated Yellow Fever virus (e.g., synthetic YFV 17D) and thereby elicit an innate immune response in the tumor cells via the activation of innate signaling receptors RIG-I, STNG, and innate immunity transcription factors IRF3, IRF7, or NFkB in tumors.

It is a further objective of the present invention to treat cancer cells by infecting them with attenuated Yellow Fever virus (e.g., synthetic YFV 17D) and thereby eliciting a pro-inflammatory immune response in the tumor.

It is a further objective of the present invention to treat cancer cells by infecting them with attenuated Yellow Fever virus (e.g., synthetic YFV 17D) and thereby recruiting pro-inflammatory white-blood cells to the tumor.

It is a further objective of the present invention to treat cancer cells by infecting them with attenuated Yellow Fever virus (e.g., synthetic YFV 17D) and thereby decreasing regulatory white-blood cells from the tumor.

It is a further objective of the present invention to pre-treat the recipient with an attenuated Yellow Fever virus (e.g., synthetic YFV 17D) to elicit an immune response before administering the virus to treat the cancer.

It is a further objective of the present invention to develop an attenuated Yellow Fever virus (e.g., synthetic YFV 17D), which would be suitable for the treatment of adenocarcinomas, and in particular, cervical cancer.

It is a further objective of the present invention to develop an attenuated Yellow Fever virus (e.g., synthetic YFV 17D), which would be suitable for the treatment of cancer cells that are positive for keratin; for example, by immunoperoxidase staining.

It is a further objective of the present invention to develop further an attenuated Yellow Fever virus (e.g., synthetic YFV 17D), which would be suitable for the treatment of cancer cells where p53 gene expression is reported to be low or absent.

It is a further objective of the present invention to develop further an attenuated Yellow Fever virus (e.g., synthetic YFV 17D), which would be suitable for the treatment of tumors where the cells are hypodiploid.

It is a further objective of the present invention to develop an attenuated Yellow Fever virus (e.g., synthetic YFV 17D), which is suitable for the treatment of lung carcinomas, and in particular, lung cancer.

It is a further objective of the present invention to develop an attenuated Yellow Fever virus (e.g., synthetic YFV 17D), which is suitable for the treatment of cancer that are hypotriploid (e.g., 64, 65, or 66 chromosome count in about 40% of cells).

It is a further objective of the present invention to develop an attenuated Yellow Fever virus (e.g., synthetic YFV 17D), which is suitable for the treatment of cancer that are have had single copies of Chromosomes N2 and N6 per cell.

It is a further objective of the present invention to develop an attenuated Yellow Fever virus (e.g., synthetic YFV 17D), which is suitable for the treatment of cancer that express the isoenzyme G6PD-B of the enzyme of the enzyme glucose-6-phosphate dehydrogenase (G6PD).

It is a further objective of the present invention to develop an attenuated Yellow Fever virus (e.g., synthetic YFV 17D), which is suitable for the treatment of melanoma.

It is a further objective of the present invention to develop an attenuated Yellow Fever virus (e.g., synthetic YFV 17D), which is suitable for the treatment of malignant cells derived from melanocytes.

It is a further objective of the present invention to develop an attenuated Yellow Fever virus (e.g., synthetic YFV 17D), which is suitable for the treatment of cancer that has MYCN oncogene amplification of at least 3-fold.

It is a further objective of the present invention to develop an attenuated Yellow Fever virus (e.g., synthetic YFV 17D), which is suitable for the treatment of breast cancer; in various embodiments it is for the treatment of triple negative breast cancer.

It is a further objective of the present invention to develop an attenuated Yellow Fever virus (e.g., synthetic YFV 17D), which is suitable for the treatment of bladder cancer.

It is a further objective of the present invention to develop an attenuated Yellow Fever virus (e.g., synthetic YFV 17D), which is suitable for the treatment of colon cancer.

It is a further objective of the present invention to develop an attenuated Yellow Fever virus (e.g., synthetic YFV 17D), which is suitable for the treatment of prostate cancer.

It is a further objective of the present invention to develop an attenuated Yellow Fever virus (e.g., synthetic YFV 17D), which is suitable for the treatment of peripheral nerve sheath tumors.

Embodiments of the present invention also provides a therapeutic composition for treating in a subject comprising the Yellow Fever virus 17D and a pharmaceutically acceptable carrier. This invention also provides a therapeutic composition for eliciting an immune response in a subject having cancer, comprising the Yellow Fever virus 17D and a pharmaceutically acceptable carrier. The invention further provides a modified host cell line specially engineered to be permissive for a Yellow Fever virus 17D that is inviable in a wild type host cell.

According to the invention, synthetic Yellow Fever virus 17D is made by transfecting synthetic viral genomes into host cells, whereby virus particles are produced. The invention further provides pharmaceutical compositions comprising synthetic Yellow Fever virus 17D which is suitable for treatment of cancer.

To further these objectives, various embodiments of the present invention provide for a method of treating a malignant tumor or reducing tumor size, comprising: administering attenuated Yellow Fever virus (YFV) to a subject in need thereof. Various embodiments of the invention provide for a method of treating a malignant tumor, comprising: administering a prime dose of attenuated YFV to a subject in need thereof; and administering one or more boost dose of attenuated YFV to the subject in need thereof. Various embodiments of the present invention provide for a method of reducing tumor size, comprising administering a prime dose of attenuated YFV to a subject in need thereof; and administering one or more boost dose of attenuated YFV to the subject in need thereof.

In various embodiments, the attenuated YFV can be YFV strain 17D vaccine (YFV 17D). In various embodiments, the attenuated YFV can be synthetic YFV strain 17D (YFV 17D). In various embodiments, the attenuated YFV can be YFV 17D-204, YFV 17DD, YFV 17D-213, codon deoptimized YFV, codon-pair deoptimized YFV, or YFV deoptimized by increasing CG or TA (or UA) dinucleotide content.

In various embodiments, the prime dose can be administered subcutaneously, intramuscularly, intradermally, intranasally, or intravenously. In various embodiments, the one or more boost dose can be administered intratumorally or intravenously. In various embodiments, a first of the one or more boost dose can be administered about 2 weeks after one prime dose, or if more than one prime dose then about 2 weeks after the last prime dose.

In various embodiments, the subject can have cancer.

In various embodiments, the prime dose can be administered when the subject does not have cancer. In various embodiments, the subject can be at a higher risk of developing cancer.

In various embodiments, the one or more boost dose can be administered about every 1, 2, 3, 4, 5, 6, 7, 8, 9 or 10 years after the prime dose when the subject does not have cancer. In various embodiments, the subject can be subsequently diagnosed with cancer and the one or more boost dose can be administered after the subject is diagnosed with cancer.

In various embodiments, the method can further comprise administering a PD-1 inhibitor or a PD-L1 inhibitor. In various embodiments, the PD-1 inhibitor can be an anti-PD1 antibody. In various embodiments, the anti-PD1 antibody can be selected from the group consisting of pembrolizumab, nivolumab, pidilizumab, AMP-224, AMP-514, spartalizumab, cemiplimab, AK105, BCD-100, BI 754091, JS001, LZM009, MGA012, Sym021, TSR-042, MGD013, AK104, XmAb20717, tislelizumab, and combinations thereof. In various embodiments, the PD-1 inhibitor can be selected from the group consisting of PF-06801591, anti-PD1 antibody expressing pluripotent killer T lymphocytes (PIK-PD-1), autologous anti-EGFRvIII 4SCAR-IgT cells, and combinations thereof. In various embodiments, the PD-L1 inhibitor can be an anti-PD-L1 antibody. In various embodiments, the anti-PD-L1 antibody can be selected from the group consisting of BGB-A333, CK-301, FAZ053, KN035, MDX-1105, MSB2311, SHR-1316, atezolizumab, avelumab, durvalumab, BMS-936559, CK-301, and combinations thereof. In various embodiments, the anti-PD-L1 inhibitor can be M7824.

In various embodiments, treating the malignant tumor can decrease the likelihood of recurrence of the malignant tumor. In various embodiments, treating the malignant tumor can decrease the likelihood of having a second cancer that is different from the malignant tumor. In various embodiments, if the subject develops a second cancer that is different from the malignant tumor, the treatment of the malignant tumor can result in slowing the growth of the second cancer. In various embodiments, after remission of the malignant tumor, if the subject develops a second cancer that is different from the malignant tumor, the treatment of the malignant tumor can result in slowing the growth of the second cancer. In various embodiments, treating the malignant tumor can stimulate an inflammatory immune response in the tumor. In various embodiments, treating the malignant tumor can recruit pro-inflammatory cells to the tumor. In various embodiments, treating the malignant tumor can stimulate an anti-tumor immune response.

In various embodiments, the malignant tumor can decrease a solid tumor. In various embodiments, the malignant tumor can decrease selected from a group consisting of glioma, neuroblastoma, glioblastoma multiforme, adenocarcinoma, medulloblastoma, mammary carcinoma, prostate carcinoma, colorectal carcinoma, hepatocellular carcinoma, bladder cancer, prostate cancer, lung carcinoma, bronchial carcinoma, epidermoid carcinoma, and melanoma.

In various embodiments, the attenuated YFV can decrease administered intratumorally, intravenously, intracerebrally, intramuscularly, intraspinally or intrathecally.

In various embodiments, administering the attenuated YFV can cause cell lysis in the tumor cells.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 shows an exemplary treatment protocol.

FIG. 2A-2C depicts the immunogenicity of synthetic YFV 17D in mice. FIG. 2A depicts the neutralizing antibody titers in serum collected from C57BL/6 mice vaccinated on day 0 and 21 with 5×10⁶ PFU of synthetic YFV 17D. Sera were collected on days 0, 21, and 35 and tested for neutralizing antibodies using a plaque-reduction-neutralization 50% (PRNT50) test. After the initial vaccination all mice seroconverted (PRNT50≥32). The mean PRNT50 titer did not increase significantly from day 21 (243.2) to day 35 (240.0) indicating the induction of sterilizing immunity that prevented replication of YFV 17D after the boosting dose. FIG. 2B depicts the neutralizing antibody titers in serum collected from BALB/c mice vaccinated on day 0 and 21 with 5×10⁶ PFU of synthetic YFV 17D. Sera were collected on days 0, 21, and 35 and tested for neutralizing antibodies using a plaque-reduction-neutralization 50% (PRNT50) test. After the initial vaccination all mice seroconverted (PRNT50≥32). At 2 weeks post-boost, the mean PRNT50 titer increased from 44.8 to 195.2, a significant increase (p=0.01; Paired t-test). FIG. 2C depicts the neutralizing antibody titers in serum collected from DBA/2 mice vaccinated on day 0 and 21 with 5×10⁶ PFU of synthetic YFV 17D. Sera were collected on days 0, 21, and 35 and tested for neutralizing antibodies using a plaque-reduction-neutralization 50% (PRNT50) test. After the initial vaccination all mice seroconverted (PRNT50≥32). The mean PRNT50 titer did not increase significantly from day 21 (192) to day 35 (160.0) indicating the induction of sterilizing immunity that prevented replication of YFV 17D after the boosting dose.

FIG. 3A-3B depicts efficacy of synthetic YFV 17D in treating implanted syngeneic B16 melanoma cells in C57BL/6 mice vaccinated on days 0 and 21, implanted on day 38 and then treated 8 times with 10⁷ PFU delivered on days 49, 51, 53, 56, 69, 71, 76, and 78. FIG. 3A depicts average tumor volume (in mm³) over time in vaccinated C57BL/6 mice implanted with 10⁵ B16 cells delivered subcutaneously into the right flank in a volume of 100 μl and either mock-treated with 0.2% BSA MEM (n=10) or treated with 10⁷ PFU of synthetic YFV 17D (n=10). FIG. 3B depicts survival and was calculated using a humane early end point of ≥1,000 mm³ tumor volume in vaccinated C57BL/6 mice implanted with 10⁵ B16 cells delivered subcutaneously into the right flank in a volume of 100 μl and either mock-treated (n=10) or treated with 10⁷ PFU of synthetic YFV 17D (n=10).

FIG. 4A-4B depicts efficacy of synthetic YFV 17D in treating implanted syngeneic EMT-6 triple-negative breast cancer cells in BALB/C mice vaccinated on days 0 and 21, implanted on day 37, then treated 9 times with 10⁷ PFU of synthetic YFV 17D delivered on days 40, 42, 44, 46, 49, 51, 58, 65, and 67. FIG. 4A depicts average tumor volume (in mm³) over time in BALB/C mice implanted with 10⁴ EMT-6 cells delivered subcutaneously into the abdominal fat-pad in a volume of 100 μl and either mock-treated (n=10) or treated with 10⁷ PFU of synthetic YFV 17D (n=10). FIG. 4B depicts survival and was calculated using a humane early end point of ≥500 mm³ tumor volume in BALB/C mice implanted with 10⁴ EMT-6 cells delivered subcutaneously into the abdominal fat-pad in a volume of 100 μl and either mock-treated (n=10) or treated with 10⁷ PFU of synthetic YFV 17D (n=10).

FIG. 5A-5B depicts efficacy of synthetic YFV 17D in treating implanted syngeneic CCL53.1 melanoma cells in DBA/2 mice vaccinated on days 0 and 21, implanted on day 45, then treated 9 times with 10⁷ PFU of synthetic YFV 17D delivered on days 51, 53, 56, 58, 60, 63, 65, 72, and 79. FIG. 5A depicts average tumor volume (in mm³) over time in DBA/2 mice implanted with 10⁵ DBA/2 cells delivered subcutaneously into the right flank in a volume of 100 μl and either mock-treated with 0.2% BSA MEM (n=8) or treated with 10⁷ PFU of synthetic YFV 17D (n=8). FIG. 5B depicts survival and was calculated using a humane early end point of ≥1,000 mm³ tumor volume in DBA/2 mice implanted with 10⁵ CCL53.1 cells delivered subcutaneously into the right flank in a volume of 100 μl and either mock-treated (n=8) or treated with 10⁷ PFU of synthetic YFV 17D (n=8).

FIG. 6 depicts neutralizing antibody titers (PRNT50) from vaccination of DBA/2 mice with YFV 17D. DBA/2 mice (n=8) were vaccinated with 5×10⁶ PFU of YFV 17D on days 0 and 21 with sera collected for titration on days 0, 21, and 35.

FIG. 7A-7C depicts efficacy of YFV 17D in treating CCL-53.1 melanoma in DBA/2 mice. Efficacy of treatment was followed for 60 days post-implantation (DPI) in DBA/2 mice implanted with 10⁵ CCL-53.1 cells and injected intratumorally 9 times with 10⁷ PFU YFV 17D. A) Median tumor size (mm³) was reduced in mice vaccinated and treated with YFV 17D compared to mock treated controls. B) Median tumor size (% compared to starting tumor size) was also reduced in treated animals. C) Survival (<1,000 mm³) was increased in treated mice compared to mock-treated controls.

FIG. 8A-8B depicts efficacy of synthetic YFV 17D treatment in providing lasting immunity against subsequence challenge. BALB/C mice were vaccinated on days 0 and 21, implanted on day 37, then treated 9 times with 10⁷ PFU of synthetic YFV 17D delivered on days 40, 42, 44, 46, 49, 51, 58, 65, and 67. Half of the mice were cured of the EMT-6 tumors implanted into their fat pads, with no apparent tumor on day 88. The cured mice were challenged on day 88 with 10⁴ EMT-6 delivered subcutaneously in a volume of 100 μl into the right flank and followed daily for tumor growth. FIG. 8A depicts average tumor volume (in mm³) over time in challenged BALB/C mice. FIG. 8B depicts the percentage of mice previously cured with synthetic YFV 17D treatment (n=3) or naïve control mice (n=8) with detectable tumors post-challenge with 10⁴ EMT-6 cells.

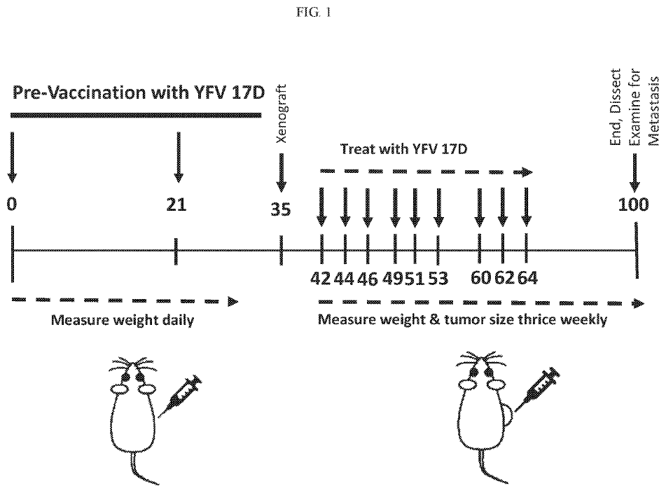

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, N.Y. 2006); and Sambrook and Russel, Molecular Cloning: A Laboratory Manual 4^{th} ed., Cold Spring Harbor Laboratory Press (Cold Spring Harbor, N.Y. 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

A “subject” means any animal or artificially modified animal. Animals include, but are not limited to, humans, non-human primates, cows, horses, sheep, pigs, dogs, cats, rabbits, ferrets, rodents such as mice, rats and guinea pigs, and birds. Artificially modified animals include, but are not limited to, SCID mice with human immune systems, outbred or inbred strains of laboratory mice, and athymic nude mice. In a preferred embodiment, the subject is a human. Preferred embodiments of birds are domesticated poultry species, including, but not limited to, chickens, turkeys, ducks, and geese.

### Oncolytic Virus Composition and Pharmaceutical Compositions

Embodiments of the present invention provide for an attenuated Yellow Fever virus. Various embodiments of the present invention provide for a pharmaceutical composition comprising an attenuated Yellow Fever virus and a pharmaceutical acceptable carrier or excipient. In various embodiments, the pharmaceutical acceptable carrier or excipient is sorbitol or gelatin, which can be used as stabilizers. In various embodiments, the composition comprising the attenuated Yellow Fever virus (e.g., vaccine preparation) can be lyophilized and kept under cold-chain conditions.

In various embodiments, the pharmaceutical acceptable carrier or excipient is particularly adapted for delivery of the attenuated Yellow Fever virus for cancer treatment; for example, to enhance delivery to the tumor site. Examples of these carriers include but are not limited to carbon nanotube, layered double hydroxide (LDH), iron oxide nanoparticles, mesoporous silica nanoparticles (MSN), polymeric nanoparticles, liposomes, micelle, protein nanoparticles, and dendrimer.

The attenuated Yellow Fever virus is one which does not cause, or has less than a 0.01% chance of causing Yellow Fever in a mammalian subject and in particular in a human subject.

In various embodiments, the attenuated Yellow Fever virus is Yellow Fever virus (YFV) 17D vaccine (e.g., UniProtKB—P03314 (POLG_YEFV1)).

The attenuated live YFV 17D vaccine strain is derived from a wild-type YF virus (the Asibi strain) isolated in Ghana in 1927 and attenuated by serial passages in chicken embryo tissue culture. Two substrains of the 17D vaccine virus are currently used for vaccine production in embryonated chicken eggs, namely 17D-204 and 17DD. Some vaccines are also prepared from a distinct substrain of 17D-204 (17D-213). Thus, in various embodiments, the attenuated YFV 17D is YFV 17D-204, YFV 17DD, or YFV 17D-213.

In various embodiments the Yellow Fever virus 17D vaccine (and its substrains) is a synthetic YFV 17D. The synthetic YFV 17D and synthetic YFV 17D substrains have the same viral genome as the live attenuated YFV 17D and live attenuated YFV 17D substrains, respectively.

Various embodiments of the invention provide an attenuated YFV virus, which comprises a modified viral genome containing nucleotide substitutions engineered in one or multiple locations in the genome, wherein the substitutions introduce a plurality of synonymous codons into the genome (e.g., codon deoptimization) and/or a change of the order of existing codons for the same amino acid (change of codon pair utilization (e.g., codon-pair deoptimization)). In both cases, the original, vaccine strain amino acid sequences are retained.

Accordingly, various embodiments of the invention provide for a codon deoptimized yellow fever virus.

In various embodiments, the codon deoptimized yellow fever virus comprises at least 10 deoptimized codons in a protein coding sequence, wherein the at least 10 deoptimized codons are each a synonymous codon less frequently used in the yellow fever virus. In various embodiments, the codon deoptimized yellow fever virus comprises at least 20, 30, 40, 50, 60, 70, 80, 90, 100, 125, 150, 200, 250, 300, 400, 500, 600, 700, 800, 900, or 1000 deoptimized codons in a protein coding sequence, wherein the at least 20, 30, 40, 50, 60, 70, 80, 90, 100, 125, 150, 200, 250, 300, 400, 500, 600, 700, 800, 900, or 1000 deoptimized codons are each a synonymous codon less frequently used in the yellow fever virus. The synonymous codon less frequently used in the yellow fever virus is a codon that encodes the same amino acid, but the codon is an unpreferred codon by the yellow fever virus for the amino acid.

TABLE 1
Yellow Fever Virus (17D Strain) Codon Usage

| Amino | | # of | Amino | | # of | Amino | | # of | Amino | | # of |
|---|---|---|---|---|---|---|---|---|---|---|---|
| acid | Codon | Use | acid | Codon | Use | acid | Codon | Use | acid | Codon | Use |
| Phe | UUU | 64 | Ser | UCU | 44 | Tyr | UAU | 35 | Cys | UGU | 32 |
| | UAC | 52 | | UCC | 42 | | UAC | 44 | | UGC | 32 |
| Leu | UUA | 8 | | UCA | 56 | Ochre | UAA | 1 | Opal | UGA | 0 |
| | UUG | 72 | | UCG | 8 | Amber | UAG | 0 | Trp | UGG | 85 |
| Leu | CUU | 46 | Pro | CCU | 40 | His | CAU | 50 | Arg | CGU | 12 |
| | CUC | 49 | | CCC | 28 | | CAC | 32 | | CGC | 25 |
| | CUA | 37 | | CCA | 56 | Gln | CAA | 42 | | CGA | 12 |
| | CUG | 100 | | CCG | 12 | | CAG | 51 | | CGG | 11 |
| Ile | AUU | 63 | Thr | ACU | 53 | Asn | AAU | 56 | Ser | AGU | 34 |
| | AUC | 69 | | ACC | 51 | | AAC | 68 | | AGC | 31 |
| | AUA | 44 | | ACA | 73 | Lys | AAA | 92 | Arg | AGA | 67 |
| Met | AUG | 129 | | ACG | 21 | | AAG | 101 | | AGG | 83 |
| Val | GUU | 69 | Ala | GCU | 83 | Asp | GAU | 70 | Gly | GGU | 36 |
| | GUC | 69 | | GCC | 80 | | GAC | 88 | | GGC | 68 |
| | GUA | 16 | | GCA | 58 | Glu | GAA | 108 | | GGA | 124 |
| | GUG | 132 | | GCG | 23 | | GAG | 102 | | GGG | 73 |

Codon usage for the yellow fever virus, 17D strain, long open reading frame of 10,233 nucleotides (3411 codons excluding the termination codon). Data from Rice et al. (1985)

In various embodiments, the codon deoptimized yellow fever virus comprises a at least 10 deoptimized codons in a protein coding sequence, wherein the at least 10 deoptimized codons are each a synonymous codon less frequently used in the viral host, such as in humans. In various embodiments, the codon deoptimized yellow fever virus comprises a at least 20, 30, 40, 50, 60, 70, 80, 90, 100, 125, 150, 200, 250, 300, 400, 500, 600, 700, 800, 900, or 1000 deoptimized codons in a protein coding sequence, wherein the at least 20, 30, 40, 50, 60, 70, 80, 90, 100, 125, 150, 200, 250, 300, 400, 500, 600, 700, 800, 900, or 1000 deoptimized codons are each a synonymous codon less frequently used in the viral host, such as humans. The synonymous codon less frequently used in in the viral host is a codon that encodes the same amino acid, but the codon is an unpreferred codon by that viral host for the amino acid. The synonymous codon less frequently used in humans is a codon that encodes the same amino acid, but the codon is an unpreferred codon by humans for the amino acid.

In various embodiments, the codon deoptimized yellow fever virus has the same amino acid sequence as YFV 17D, YFV 17D-204, YFV 17DD, or YFV 17D-213. In various embodiments, the codon deoptimized yellow fever virus has up to 1, 2, 3, 4 or 5 amino acid changes as compared to the amino acid sequence as YFV 17D, YFV 17D-204, YFV 17DD, or YFV 17D-213. An amino acid change can be a different amino acid, a deletion of an amino acid, or an addition of an amino acid.

Methods of codon deoptimization are described in International Application No. PCT/US2005/036241, the contents of which are herein incorporated by reference.

Various embodiments of the invention provide for a codon-pair deoptimized (CPD) yellow fever virus.

In various embodiments, the codon-pair deoptimized yellow fever virus comprises a reduction in codon-pair bias (CPB) as compared to the yellow fever virus before codon-pair deoptimization of the yellow fever virus. Thus, the codon-pair deoptimized yellow fever virus comprises rearranging existing codons in a protein encoding sequence. Rearranging existing codons in a protein encoding sequence comprises substituting a codon pair with a codon pair that has a lower codon-pair score.

As such, it comprises recoded protein encoding sequences wherein each sequence has existing synonymous codons from its parent protein-encoding sequence in a rearranged order and has a CPB less than the CPB of the parent protein-encoding sequence from which it is derived.

In some embodiments, a subset of codon pairs is substituted by rearranging a subset of synonymous codons. In other embodiments, codon pairs are substituted by maximizing the number of rearranged synonymous codons. It is noted that while rearrangement of codons leads to codon-pair bias that is reduced (made more negative) for the virus coding sequence overall, and the rearrangement results in a decreased codon pair scores (CPS) at many locations, there may accompanying CPS increases at other locations, but on average, the codon pair scores, and thus the CPB of the modified sequence, is reduced.

In various embodiments, the CPB is reduced by at least 0.01, at least 0.02, at least 0.03, at least 0.04, at least 0.05, at least 0.10, at least 0.15, at least 0.20, at least 0.25, at least 0.30, at least 0.35, at least 0.40, at least 0.45 or at least 0.50.

In various embodiments, the codon pair bias is based on codon pair usage in yellow fever virus. In various embodiments, the codon pair bias is based on codon pair usage in humans.

In various embodiments, the codon-pair deoptimized yellow fever virus has the same amino acid sequence as YFV 17D, YFV 17D-204, YFV 17DD, or YFV 17D-213. In various embodiments, the codon-pair deoptimized yellow fever virus has up to 1, 2, 3, 4, or 5 amino acid changes as compared to the amino acid sequence as YFV 17D, YFV 17D-204, YFV 17DD, or YFV 17D-213. An amino acid change can be a different amino acid, a deletion of an amino acid, or an addition of an amino acid.

Method of codon-pair deoptimization are described in International Patent Application No. PCT/US2008/058952, the contents of which are herein incorporated by reference.

Various embodiments of the invention provide for a deoptimized yellow fever virus wherein the frequency of the CG and/or TA (or UA) dinucleotide content is altered. In various embodiments, the CpG dinucleotide content in the deoptimized YFV is increased. In various embodiments, the UpA dinucleotide content in the deoptimized YFV is increased.

In various embodiments, the deoptimized yellow fever virus has the same amino acid sequence as YFV 17D, YFV 17D-204, YFV 17DD, or YFV 17D-213. In various embodiments, the deoptimized yellow fever virus has up to 1, 2, 3, 4, or 5 amino acid changes as compared to the amino acid sequence as YFV 17D, YFV 17D-204, YFV 17DD, or YFV 17D-213. An amino acid change can be a different amino acid, a deletion of an amino acid, or an addition of an amino acid.

Method of altering CG and/or TA (or UA) dinucleotide content are described in International Patent Application No. PCT/US2008/058952, the contents of which are herein incorporated by reference.

The attenuated YFV of this invention, and particularly, the synthetic YFV 17D is useful in prophylactic and therapeutic compositions for reducing tumor size and treating malignant tumors in various organs, such as: breast, colon, bronchial passage, epithelial lining of the gastrointestinal, upper respiratory and genito-urinary tracts, liver, prostate, the brain, or any other human tissue. In various embodiments, the modified YFV of the present invention are useful for reducing the size of solid tumors and treating solid tumors. In particular embodiments, the tumors treated or reduced in size is glioma, glioblastoma, adenocarcinoma, melanoma, or neuroblastoma. In various embodiments, the tumor is a triple-negative breast cancer.

The pharmaceutical compositions of this invention may further comprise other therapeutics for the prophylaxis of malignant tumors. For example, the modified YFV of this invention may be used in combination with surgery, radiation therapy and/or chemotherapy. Furthermore, one or more modified YFV may be used in combination with two or more of the foregoing therapeutic procedures. Such combination therapies may advantageously utilize lower dosages of the administered therapeutic agents, thus avoiding possible toxicities or adverse effects associated with the various monotherapies.

The pharmaceutical compositions of this invention comprise a therapeutically effective amount of one or more modified YFV according to this invention, and a pharmaceutically acceptable carrier. By “therapeutically effective amount” is meant an amount capable of causing lysis of the cancer cells to cause tumor necrosis. By “pharmaceutically acceptable carrier” is meant a carrier that does not cause an allergic reaction or other untoward effect in patients to whom it is administered.

Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the modified viral chimeras.

The compositions of this invention may be in a variety of forms. These include, for example, liquid dosage forms, such as liquid solutions, dispersions or suspensions, injectable and infusible solutions. The preferred form depends on the intended mode of administration and prophylactic or therapeutic application. The preferred compositions are in the form of injectable or infusible solutions.

Recombinant modified YFV can be synthesized by well-known recombinant DNA techniques. Any standard manual on DNA technology provides detailed protocols to produce the modified viral chimeras of the invention.

This invention further provides a method of synthesizing any of the viruses described herein, the method comprising (a) identifying the target virus to be synthesized, (b) completely sequencing the target virus or locating the sequence on a publicly or privately available database, (c) de novo synthesis of DNA containing the coding and noncoding region of the genome as a complete plasmid known as an “infectious clone” or as individual pieces of synthetic DNA that can be joined using overlapping PCR. In further embodiments, the entire genome is substituted with the synthesized DNA. In still further embodiments, a portion of the genome is substituted with the synthesized DNA. In yet other embodiments, said portion of the genome is the capsid coding region.

Prophylactic and Therapeutic Cancer treatments

The present invention relates to the production of Yellow Fever viruses and compositions comprising these Yellow Fever viruses that can be used as oncolytic therapy to treat different tumor types and methods of treating tumors and cancer by administering the attenuated YFV virus, such as, the attenuated (including attenuation by deoptimization) YFV 17D, YFV 17D-204, YFV 17DD, or YFV 17D-213, and particularly, the synthetic YFV 17D described herein.

### Treatment of Existing Cancer

Various embodiments of the present invention provide for a method of inducing an oncolytic effect on a tumor or cancer cell. In various embodiments, this type of treatment can be made when a subject has been diagnosed with cancer. The method comprises administering attenuated YFV to a subject in need thereof. The attenuated YFV can be provided and administered in a composition comprising a pharmaceutical acceptable carrier or excipient as provided herein.

In various embodiments, the attenuated YFV is YFV 17D vaccine having the sequence provided as UniProtKB—P03314 (POLG_YEFV1) as of the filing date of the present.

In various embodiments, the attenuated YFV is YFV 17D-204, YFV 17DD, or YFV 17D-213.

In various embodiments the Yellow Fever virus 17D vaccine (and its substrains) is a synthetic YFV 17D. The synthetic YFV 17D and synthetic YFV 17D substrains have the same viral genome as the live attenuated YFV 17D and live attenuated YFV 17D substrains, respectively.

In various embodiments the attenuated Yellow Fever virus is a codon deoptimized YFV, codon-pair deoptimized YFV, or YFV deoptimized by increasing CG or TA (or UA) dinucleotide content as described herein.

In various embodiments, inducing an oncolytic effect on a malignant tumor results in treating the malignant tumor.

In various embodiments, the method of treatment further comprises administering a PD-1 inhibitor. In other embodiments, the method of treatment further comprises administering a PD-L1 inhibitor. In still other embodiments, the method of treatment further comprises administering both an PD-1 inhibitor and a PD-L1 inhibitor.

In various embodiments, the PD-1 inhibitor is an anti-PD1 antibody. In various embodiments, the PD-L1 inhibitor is an anti-PD-L1 antibody. Examples of PD-1 inhibitors and PD-L1 inhibitors that are used are provided herein.

In various embodiments, the treatment of the malignant tumor decreases the likelihood of recurrence of the malignant tumor. It can also decrease the likelihood of having a second cancer that is different from the malignant tumor. If the subject develops a second cancer that is different from the malignant tumor and the treatment of the malignant tumor results in slowing the growth of the second cancer. In some embodiments, after remission of the malignant tumor, the subject develops a second cancer that is different from the malignant tumor and the treatment of the malignant tumor results in slowing the growth of the second cancer.

### Prime-Boost Treatments

Various embodiments of the present invention provide for a method of eliciting an immune response and inducing an oncolytic effect on a tumor or cancer cell, using a prime-boost-type treatment regimen. In various embodiments, eliciting the immune response and inducing an oncolytic effect on the tumor or cancer cell results in treating a malignant tumor.

A prime dose of the attenuated YFV, and particularly, the synthetic YFV 17D of the present invention is administered to elicit an initial immune response. Thereafter, a boost dose of attenuated YFV, and particularly, the synthetic YFV 17D of the present invention is administered to induce oncolytic effects on the tumor and/or to elicit an immune response comprising oncolytic effect against the tumor.

In various embodiments, the method comprises administering a prime dose of an attenuated YFV, and particularly, the synthetic YFV 17D to a subject in need thereof; and administering one or more boost dose of an attenuated YFV, and particularly, the synthetic YFV 17D to the subject in need thereof.

In various embodiments, the attenuated YFV is YFV 17D-204, YFV 17DD, or YFV 17D-213. In various embodiments the attenuated YFV is a codon deoptimized YFV, codon-pair deoptimized YFV, or YFV deoptimized by increasing CG or TA (or UA) dinucleotide content as described herein. In various embodiments the attenuated YFV is a codon deoptimized YFV 17D, YFV 17D-204, YFV 17DD, or YFV 17D-213, codon-pair deoptimized YFV 17D, YFV 17D-204, YFV 17DD, or YFV 17D-213, or YFV 17D, YFV 17D-204, YFV 17DD, or YFV 17D-213 deoptimized by increasing CG or TA (or UA) dinucleotide content as described herein.

In various embodiments, the prime dose is administered subcutaneously, intramuscularly, intradermally, intranasally or intravenously.

In various embodiments, the one or more boost dose is administered intratumorally, intravenously, intrathecally or intraneoplastically (directly into the tumor). A preferred mode of administration is directly to the tumor site.

The timing between the prime and boost dosages can vary, for example, depending on the type of cancer, the stage of cancer, and the patient's health. In various embodiments, the first of the one or more boost dose is administered about 2 weeks after the prime dose. That is, the prime dose is administered and about two weeks thereafter, the boost dose is administered.

In various embodiments, the one or more boost dose is administered about 1 week after a prime dose. In various embodiments, the one or more boost dose is administered about 2 weeks after a prime dose. In various embodiments, the one or more boost dose is administered about 3 weeks after a prime dose. In various embodiments, the one or more boost dose is administered about 4 weeks after a prime dose. In various embodiments, 2, 3, 4, 5, 6, 7, 8, 9, 10, 11, 12, 13, 14, or 15 boost doses are administered. In various embodiments, 1-5, 5-10, 10-15, 15-20, 20-25, 25-30, 30-35, 35-40, 40-45 or 45-50 boost doses are administered. In various embodiments, the intervals between the boost doses can be 1, 2, 3, 4, 5, 6, 7, 8, 9, or 10 weeks. In additional embodiments, the intervals between the boost doses can be 1, 2, 3, 4, 5, 6, 7, 8, 9, 10, 11 or 12 months. As a non-limiting example, the prime dose can be administered, about two weeks thereafter a first boost dose can be administered, about one month after the first boost dose, a second boost dose can be administered, about 6 months after the second boost dose, a third boost dose can be administered. As another non-limiting example, the prime dose can be administered, about two weeks thereafter 10 boost doses are administered at one dose per week. As another non-limiting example, the prime dose can be administered, about two weeks thereafter a first boost dose can be administered, about six months after the first boost dose, a second boost dose can be administered, about 12 months after the second boost dose, a third boost dose can be administered. In further embodiments, additional boost dosages can be periodically administered; for example, every year, every other year, every 5 years, every 10 years, etc.

In various embodiments, the dosage amount can vary between the prime and boost dosages. As a non-limiting example, the prime dose can contain fewer copies of the virus compare to the boost dose.

In other embodiments, the route of administration can vary between the prime and the boost dose. In a non-limiting example, the prime dose can be administered subcutaneously, and the boost dose can be administered via injection into the tumor; for tumors that are in accessible, or are difficult to access, the boost dose can be administered intravenously.

In various embodiments, the treatment further comprises administering a PD-1 inhibitor. In other embodiments, the treatment further comprises administering a PD-L1 inhibitor. In still other embodiments, the treatment further comprises administering both an PD-1 inhibitor and a PD-L1 inhibitor. In particular embodiments, the PD-1 inhibitor, the PD-L1 inhibitor, or both are administered during the treatment (boost) phase, and not during the priming phase.

In various embodiments, the PD-1 inhibitor is an anti-PD1 antibody. In various embodiments, the PD-L1 inhibitor is an anti-PD-L1 antibody. Examples of PD-1 inhibitors and PD-L1 inhibitors are provided herein.

### Prime-Boost Treatment Before Having Cancer

Various embodiments of the present invention provide for a method of eliciting an immune response in a subject who does not have cancer and inducing an oncolytic effect on a tumor or cancer cell if and when the tumor or cancer cell develops in the subject. The method uses a prime-boost-type treatment regimen. In various embodiments, eliciting the immune response and inducing an oncolytic effect on the tumor or cancer cell results in treating a malignant tumor if and when the subject develops cancer.

A prime dose of attenuated YFV, and particularly, the synthetic YFV 17D of the present invention is administered to elicit an initial immune response when the subject does not have cancer or when the subject is not believed to have cancer. The latter may be due to undetectable or undetected cancer.

Thereafter, in some embodiments, a boost dose of attenuated YFV, and particularly, the synthetic YFV 17D of the present invention is administered periodically to continue to elicit the immune response. For example, a boost dose can be administered about every 1, 2, 3, 4, 5, 6, 7, 8, 9, or 10 years. In particular embodiments, the boost dose can be administered about every 5 years.

Alternatively, in other embodiments, a boost dose of attenuated YFV, and particularly, the synthetic YFV 17D of the present invention is administered after the subject is diagnosed with cancer. For example, once the subject is diagnosed with cancer, a treatment regimen involving the administration of a boost dose can be started shortly thereafter to induce oncolytic effects on the tumor and/or to elicit an immune response comprising an oncolytic effect against the tumor. In further embodiments, additional boost doses can be administered to continue to treat the cancer.

In various embodiments, the attenuated YFV is YFV 17D-204, YFV 17DD, or YFV 17D-213. In various embodiments the attenuated YFV is a codon deoptimized YFV, codon-pair deoptimized YFV, or YFV deoptimized by increasing CG or TA (or UA) dinucleotide content as described herein. In various embodiments the attenuated YFV is a codon deoptimized YFV 17D, YFV 17D-204, YFV 17DD, or YFV 17D-213, codon-pair deoptimized YFV 17D, YFV 17D-204, YFV 17DD, or YFV 17D-213, or YFV 17D, YFV 17D-204, YFV 17DD, or YFV 17D-213 deoptimized by increasing CG or TA (or UA) dinucleotide content as described herein.

While not wishing to be bound by any particular theory, or set regimen, it is believed that the prime dose and boost dose(s) “teach” the subject's immune system to recognize virus-infected cells. Thus, when the subject develops cancer and the boost dose is administered, the subject's immune system recognizes the virus infected cells; this time, the virus infected cells are the cancer cells. During the immune response to the virus infected cancer cells, the immune system is also primed with cancer antigens, and thus enhances the anti-cancer immunity as the immune system will also target the cells expressing the cancer antigens.

As such, in various embodiments, the treatment of the malignant tumor decreases the likelihood of recurrence of the malignant tumor. It can also decrease the likelihood of having a second cancer that is different from the malignant tumor. If the subject develops a second cancer that is different from the malignant tumor and the treatment of the malignant tumor results in slowing the growth of the second cancer. In some embodiments, after remission of the malignant tumor, the subject develops a second cancer that is different from the malignant tumor and the treatment of the malignant tumor results in slowing the growth of the second cancer.

One can think of the prime and boost doses as an anti-cancer vaccine, preparing the immune system to target treated tumor cells when cancer develops.

In various embodiments, the prime dose is administered subcutaneously, intramuscularly, intradermally, intranasally or intravenously.

In various embodiments, the one or more boost dose, when it is administered to a subject who does not have cancer, or is not suspected to have cancer, it is administered subcutaneously, intramuscularly, intradermally, intranasally or intravenously.

In various embodiments, the one or more boost dose, when it is administered to a subject who had been diagnosed with cancer, it is administered intratumorally, intravenously, intrathecally or intraneoplastically (directly into the tumor). A preferred mode of administration is directly to the tumor site.

The timing between the prime and boost dosages can vary, for example, depending on the type of cancer, the stage of cancer, and the patient's health. In various embodiments, the first of the one or more boost dose is administered about every 1, 2, 3, 4, 5, 6, 7, 8, 9, or 10 years after the prime dose, if the subject does not have cancer or is not suspected to have cancer. In particular embodiments, the boost dose is administered about every 5 years.

In various embodiments, for example, when the subject is diagnosed with cancer the one or more boost dose is administered after the diagnosis of cancer. In various embodiments, 2, 3, 4, or 5 boost doses are administered. In various embodiments, 2, 3, 4, 5, 6, 7, 8, 9, or 10 boost doses are administered. In various embodiments, the intervals between the boost doses can be 1, 2, 3, 4, 5, 6, 7, 8, 9, or 10 weeks. In additional embodiments, the intervals between the boost doses can be 1, 2, 3, 4, 5, 6, 7, 8, 9, 10, 11 or 12 months. As a non-limiting example, the prime dose can be administered, about five years thereafter, a first boost dose can be administered, about one year after the first boost dose, the subject is diagnosed with cancer, and a second boost dose can be administered, about 2 weeks after the second boost dose, a third boost dose can be administered, about 2 weeks after the third boost dose, a fourth boost dose can be administered, and about 1 month after the fourth boost dose a fifth boost dose can be administered. Once the cancer is determined to be in remission, additional periodic boost doses can be administered; for example, every 6 months, every year, every 2, years, every 3, years, every 4 years or every 5 years.

In various embodiments, the dosage amount can vary between the prime and boost dosages. As a non-limiting example, the prime dose can contain fewer copies of the virus compare to the boost dose.

In other embodiments, the route of administration can vary between the prime and the boost dose. In a non-limiting example, the prime dose can be administered subcutaneously, and the boost dose can be administered via injection into the tumor (when the subject has cancer); for tumors that are in accessible, or are difficult to access, the boost dose can be administered intravenously.

In various embodiments, subjects that receive these treatments (e.g., prime dose before having cancer, or prime and boost doses before having cancer, and then followed by boost doses after having cancer) can be a subject who are at a higher risk of developing cancer. Examples of such subject include but are not limited to, subjects with genetic dispositions (e.g., BRCA1 or BRCA2 mutation, TP53 mutations, PTEN mutations, KRAS mutations, c-Myc mutations, any mutation deemed by the National Cancer Institute as a cancer-predisposing mutation, etc.), family history of cancer, advanced age (e.g., 40, 45, 55, 65 years or older), higher than normal radiation exposure, prolonged sun exposure, history of tobacco use (e.g., smoking, chewing), history of alcohol abuse, history of drug abuse, a body mass index >25, history of a chronic inflammatory disease(s) (e.g., inflammatory bowel diseases, ulcerative colitis, Crohn disease, asthma, rheumatoid arthritis, etc.), history of immune suppression, history of chronic infections known to have a correlation to increased cancer risk (e.g., Hepatitis C, Hepatitis B, EBV, CMV, HPV, HIV, HTLV-1, MCPyV, H. Pylori, etc.).

In various embodiments, subjects that receive these treatments (e.g., prime dose and boost dose before having cancer, or prime and boost doses before having cancer, and then followed by boost doses after having cancer) can be subjects who do not fall into the higher risk category but are prescribed the prime and boost doses by their clinician as a preventive measure for future cancer risk.

In various embodiments, the treatment further comprises administering a PD-1 inhibitor. In other embodiments, the treatment further comprises administering a PD-L1 inhibitor. In still other embodiments, the treatment further comprises administering both an PD-1 inhibitor and a PD-L1 inhibitor. In particular embodiments, the PD-1 inhibitor, the PD-L1 inhibitor, or both are administered during the treatment (boost) phase, and not during the priming phase.

In various embodiments, the PD-1 inhibitor is an anti-PD1 antibody. In various embodiments, the PD-L1 inhibitor is an anti-PD-L1 antibody. Examples of PD-1 inhibitors and PD-L1 inhibitors are provided herein.

### Inflammatory Response

In various embodiments, the administration of the Yellow Fever virus 17D of the present invention to stimulate endogenous Type-1 interferon production in the subject which provides, in part, the therapeutic efficacy.

In various embodiments, the administration of the modified viruses of the present invention to maintain a therapeutically effective amount of Type-1 interferon production in the subject which provides, in part, the therapeutic efficacy.

In still other embodiments, the administration of the modified viruses of the present invention to activate of Type I Interferon in a subject to maintain ionizing radiation and chemotherapy sensitization in the subject.

In various embodiments the administration of the modified viruses of the present invention to recruit pro-inflammatory immune cells including CD45+ Leukocytes, Neutrophils, B-cells, CD4+ T-cells, and CD8+ immune cells to the site of cancer, which provides, in part, the therapeutic efficacy.

In various embodiments the administration of the modified viruses of the present invention to decrease anti-inflammatory immune cells such as FoxP3+T-regulatory cells or M2-Macrophages from the site of cancer, which provides, in part, the therapeutic efficacy.

In various embodiments, the treatment of the malignant tumor decreases the likelihood of recurrence of the malignant tumor. It can also decrease the likelihood of having a second cancer that is different from the malignant tumor. If the subject develops a second cancer that is different from the malignant tumor and the treatment of the malignant tumor results in slowing the growth of the second cancer. In some embodiments, after remission of the malignant tumor, the subject develops a second cancer that is different from the malignant tumor and the treatment of the malignant tumor results in slowing the growth of the second cancer.

### PD-1 Inhibitors and PD-L1 Inhibitors

Examples of anti-PD1 antibodies that can be used as discussed herein include but are not limited to pembrolizumab, nivolumab, pidilizumab, AMP-224, AMP-514, spartalizumab, cemiplimab, AK105, BCD-100, BI 754091, JS001, LZM009, MGA012, Sym021, TSR-042, MGD013, AK104, XmAb20717, and tislelizumab.

Additional examples of PD-1 inhibitors include but are not limited PF-06801591, anti-PD1 antibody expressing pluripotent killer T lymphocytes (PIK-PD-1), and autologous anti-EGFRvIII 4SCAR-IgT cells.

Examples of anti-PD-L1 antibody include but are not limited to BGB-A333, CK-301, FAZ053, KN035, MDX-1105, MSB2311, SHR-1316, atezolizumab, avelumab, durvalumab, BMS-936559, and CK-301. An additional example of an anti-PD-L1 inhibitor is M7824.

### Routes of Administration

In additional to those discussed above, therapeutic oncolytic YFV 17D virus (or YFV 17D-204, YFV 17DD, or YFV 17D-213, or codon deoptimized YFV, codon-pair deoptimized YFV, or YFV deoptimized by increasing CG or TA (or UA) dinucleotide content, or codon deoptimized YFV 17D, YFV 17D-204, YFV 17DD, or YFV 17D-213, codon-pair deoptimized YFV 17D, YFV 17D-204, YFV 17DD, or YFV 17D-213, or YFV 17D, YFV 17D-204, YFV 17DD, or YFV 17D-213 deoptimized by increasing CG or TA (or UA) dinucleotide content as described herein as described herein) can be delivered intratumorally, intravenously, intrathecally or intraneoplastically (directly into the tumor). A preferred mode of administration is directly to the tumor site. The inoculum of virus applied for therapeutic purposes can be administered in an exceedingly small volume ranging between 1-10 μl.

It will be apparent to those of skill in the art that the therapeutically effective amount of YFV 17D virus (or YFV 17D-204, YFV 17DD, or YFV 17D-213, or codon deoptimized YFV, codon-pair deoptimized YFV, or YFV deoptimized by increasing CG or TA (or UA) dinucleotide content, or codon deoptimized YFV 17D, YFV 17D-204, YFV 17DD, or YFV 17D-213, codon-pair deoptimized YFV 17D, YFV 17D-204, YFV 17DD, or YFV 17D-213, or YFV 17D, YFV 17D-204, YFV 17DD, or YFV 17D-213 deoptimized by increasing CG or TA (or UA) dinucleotide content as described herein) of this invention can depend upon the administration schedule, the unit dose of YFV 17D virus (or YFV 17D-204, YFV 17DD, or YFV 17D-213, or codon deoptimized YFV, codon-pair deoptimized YFV, or YFV deoptimized by increasing CG or TA (or UA) dinucleotide content as described herein) administered, whether the YFV 17D virus (or YFV 17D-204, YFV 17DD, or YFV 17D-213, or codon deoptimized YFV, codon-pair deoptimized YFV, or YFV deoptimized by increasing CG or TA (or UA) dinucleotide content, or codon deoptimized YFV 17D, YFV 17D-204, YFV 17DD, or YFV 17D-213, codon-pair deoptimized YFV 17D, YFV 17D-204, YFV 17DD, or YFV 17D-213, or YFV 17D, YFV 17D-204, YFV 17DD, or YFV 17D-213 deoptimized by increasing CG or TA (or UA) dinucleotide content as described herein) is administered in combination with other therapeutic agents, the status and health of the patient. In various embodiments, a therapeutically effective amount of 4.74 log 10+/−2 log 10 of YFV 17D virus of this invention is administered.

The therapeutically effective amounts of oncolytic recombinant virus can be determined empirically and depend on the maximal amount of the recombinant virus that can be administered safely, and the minimal amount of the recombinant virus that produces efficient oncolysis.

Therapeutic inoculations of oncolytic attenuated YFV (or YFV 17D-204, YFV 17DD, or YFV 17D-213, or codon deoptimized YFV, codon-pair deoptimized YFV, or YFV deoptimized by increasing CG or TA (or UA) dinucleotide content, or codon deoptimized YFV 17D, YFV 17D-204, YFV 17DD, or YFV 17D-213, codon-pair deoptimized YFV 17D, YFV 17D-204, YFV 17DD, or YFV 17D-213, or YFV 17D, YFV 17D-204, YFV 17DD, or YFV 17D-213 deoptimized by increasing CG or TA (or UA) dinucleotide content as described herein), and particularly, the synthetic YFV 17D can be given repeatedly, depending upon the effect of the initial treatment regimen. Should the host's immune response to the oncolytic attenuated YFV (or YFV 17D-204, YFV 17DD, or YFV 17D-213, or codon deoptimized YFV, codon-pair deoptimized YFV, or YFV deoptimized by increasing CG or TA (or UA) dinucleotide content, or codon deoptimized YFV 17D, YFV 17D-204, YFV 17DD, or YFV 17D-213, codon-pair deoptimized YFV 17D, YFV 17D-204, YFV 17DD, or YFV 17D-213, or YFV 17D, YFV 17D-204, YFV 17DD, or YFV 17D-213 deoptimized by increasing CG or TA (or UA) dinucleotide content as described herein), and particularly, the synthetic YFV 17D administered initially limit its effectiveness, additional injections of an oncolytic modified viruses with a different modified viruses' serotype can be made. The host's immune response to attenuated YFV (or YFV 17D-204, YFV 17DD, or YFV 17D-213, or codon deoptimized YFV, codon-pair deoptimized YFV, or YFV deoptimized by increasing CG or TA (or UA) dinucleotide content, or codon deoptimized YFV 17D, YFV 17D-204, YFV 17DD, or YFV 17D-213, codon-pair deoptimized YFV 17D, YFV 17D-204, YFV 17DD, or YFV 17D-213, or YFV 17D, YFV 17D-204, YFV 17DD, or YFV 17D-213 deoptimized by increasing CG or TA (or UA) dinucleotide content as described herein), and particularly, the synthetic YFV 17D can be easily determined serologically. It will be recognized, however, that lower or higher dosages than those indicated above according to the administration schedules selected.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Immunogenicity in Immune-Competent Mice

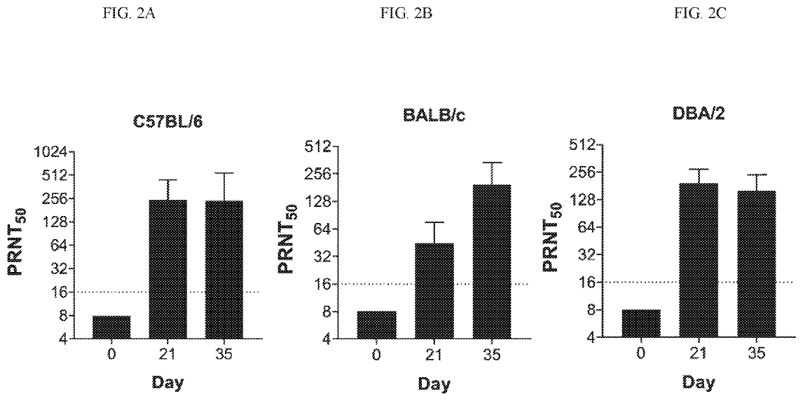

C57BL/6 mice were vaccinated on day 0 and 21 with 5×10⁶ PFU of synthetic YFV 17D (FIG. 2A). Sera were collected on days 0, 21, and 35 and tested for neutralizing antibodies using a plaque-reduction-neutralization 50% (PRNT50) test. Mice were initially seronegative for YFV 17d (PRNT50<16). After the initial vaccination all mice seroconverted (PRNT50≥32). The mean PRNT50 titer did not increase significantly from day 21 (243.2) to day 35 (240.0) indicating the induction of sterilizing immunity that prevented replication of YFV 17D after the boosting dose. BALB/c mice were vaccinated on day 0 and 21 with 5×10⁶ PFU of synthetic YFV 17D (FIG. 2B). Sera were collected on days 0, 21, and 35 and tested for neutralizing antibodies using a plaque-reduction-neutralization 50% (PRNT50) test. Mice were initially seronegative for YFV 17D (PRNT50<16). After the initial vaccination all mice seroconverted (PRNT50≥32). At 2 weeks post-boost, the mean PRNT50 titer increased from 44.8 (day 21) to 195.2 (day 35), a significant increase (p=0.01; Paired t-test). DBA/2 mice were vaccinated on day 0 and 21 with 5×10⁶ PFU of synthetic YFV 17D (FIG. 2C). Sera were collected on days 0, 21, and 35 and tested for neutralizing antibodies using a plaque-reduction-neutralization 50% (PRNT50) test. Mice were initially seronegative for YFV 17D (PRNT50<16). After the initial vaccination all mice seroconverted (PRNT50≥32). The mean PRNT50 titer did not increase significantly from day 21 (192) to day 35 (160.0) indicating the induction of sterilizing immunity that prevented replication of YFV 17D after the boosting dose. As demonstrated by the induction of neutralizing antibodies, immunity to YFV 17D was successfully induced by vaccination with synthetic YFV 17D.

### Example 2

### Oncolytic Efficacy in Immune-Competent Mice Against B16 Melanoma

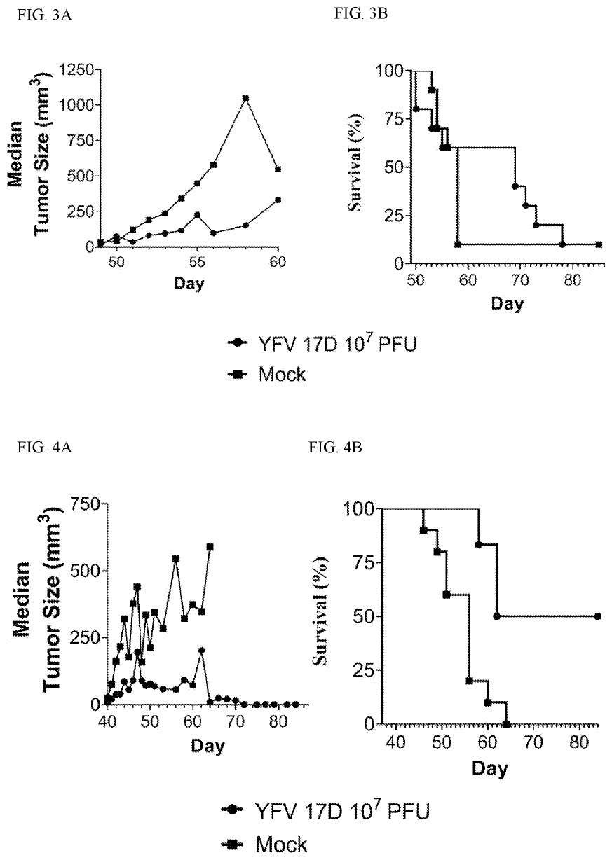

Synthetic YFV 17D was used to treat implanted syngeneic B16 melanoma cells in C57BL/6 mice vaccinated on days 0 and 21, implanted on day 38 and then treated 8 times with 10⁷ PFU delivered on days 49, 51, 53, 56, 69, 71, 76, and 78 (FIG. 3A-B). Vaccinated C57BL/6 mice were implanted with 10⁵ B16 cells delivered subcutaneously into the right flank in a volume of 100 μl and either mock-treated with 0.2% BSA MEM (n=10) or treated with 10⁷ PFU of synthetic YFV 17D (n=10). The implanted tumors were treated by direct injection with 50 μl of synthetic YFV 17D. Tumor height, width, and depth was measured using calipers each day and the tumor volume (mm³) calculated using the formula:

$$\frac{4}{3} \times π \times height \times width \times ( \frac{dept h}{2} / 1000 )$$

Tumor size was significantly reduced (FIG. 3A) in treated mice compared to mock control mice on days 52, 53, 54, 55, 57, and 58 as determined by Student's t-test comparing mean tumor sizes for each group. After day 58, most of the mock-control group had reached our humane early end-point (1,000 mm³ tumor volume) and sizes could no longer be compared. In terms of survival (using 1,000 mm³ tumor volume as a humane early end-point), the outcome in YFV 17D treated mice was greatly improved with an increase in median survival from 20 days (mock-control group) to 31 days post-implantation. As shown by survival analysis using Kaplan-Meier curves (FIG. 3B), survival in YFV 17D treated C57BL/6 mice was significantly improved compared to the mock-control group by the log-rank (Mantel-Cox) test (p=0.0141). Sample size was based on standard deviations of tumor size observed in prior experiments and chosen using GraphPad Statmate 2 to achieve sufficient statistical power (0.80).

### Example 4

### Oncolytic Efficacy Against EMT-6 Triple-Negative Breast Cancer in Immune-Competent Mice

Synthetic YFV 17D was used to treat implanted syngeneic EMT-6 triple-negative breast cancer cells in BALB/C mice vaccinated on days 0 and 21, implanted on day 37, then treated 9 times with 10⁷ PFU of synthetic YFV 17D delivered on days 40, 42, 44, 46, 49, 51, 58, 65, and 67. Vaccinated BALB/C mice were implanted with 10⁴ EMT-6 cells delivered subcutaneously into the abdominal fat-pad in a volume of 100 μl and either mock-treated (n=10) or treated with 10⁷ PFU of synthetic YFV 17D (n=10). The implanted tumors were treated by direct injection with 50 μl of synthetic YFV 17D. Tumor height, width, and depth was measured using calipers each day and the tumor volume (mm³) calculated using the formula:

$$\frac{4}{3} \times π \times height \times width \times ( \frac{dept h}{2} / 1000 )$$

Tumor size was significantly reduced (FIG. 4A) in the YFV 17D treated mice compared to the mock control group on days 41-56 as determined by Student's t-tests comparing means at each time-point. After day 23, there were too few mice remaining in the mock-control group to make statistical comparisons between the groups. Survival, as determined by the human early end-points of tumor ulceration of size ≥500 mm³ was also improved in the YFV 17D treated group compared to mock-controls. Median survival was much higher in treated (36 days) compared to mock-controls (19 days) and Kaplain-Meier curves showed improved survival in treated mice (p=<0.0001) by log-rank (Mantel-Cox) analysis. Sample size was based on standard deviations of tumor size observed in prior experiments and chosen using GraphPad Statmate 2 to achieve sufficient statistical power (0.80).

### Example 5

### Oncolytic Efficacy Against CCL-53.1 Melanoma in Immune-Competent Mice

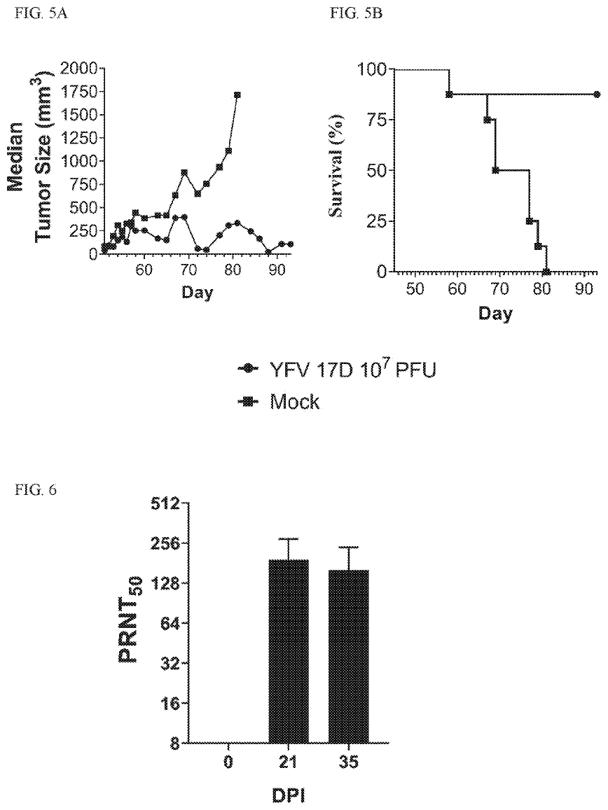

For the purpose of this study, DBA/2 mice (n=8) were initially vaccinated on days 0 and 21 with synthetic YFV 17D, then implanted with 10⁵ Clone M3, Cloudman S-91 melanoma tumor cells (ATCC CCL-53.1) on day 45, then treated 9 times with 10⁷ PFU of synthetic YFV 17D delivered on days 51, 53, 56, 58, 60, 63, 65, 72, and 79 (FIG. 5A-B). The implanted tumors were treated by direct injection with 50 μl of synthetic YFV 17D. Tumor height, width, and depth was measured using calipers each day and the tumor volume (mm³) calculated using the formula:

$$\frac{4}{3} \times π \times height \times width \times ( \frac{dept h}{2} / 1000 )$$

For mortality, early humane end-points of ≥20% weight loss, tumor ulceration, or tumor growth >1,000 mm³ were used. Sample size was based on standard deviations of tumor size observed in prior experiments and chosen using GraphPad Statmate 2 to achieve sufficient statistical power (0.80). The implanted CCL-53.1 cells responded well to oncolytic treatment with YFV 17D. Mean tumor size was significantly reduced in the treated group on days 53, 56, 60, 61, and 63-67 according to Student's t-test comparison between treated and mock-treated groups. Furthermore, median survival time was greatly increased in treated (>47 days) compared to mock controls (27.5 days). Comparison of Kaplan-Meier survival curves (FIG. 5B) also revealed significantly improved survival in treated DBA/2 mice compared the mock controls (p=0.0004) by log-rank (Mantel-Cox) test.

Melanoma can be modeled well in DBA/2 mice using CCL53.1 cell implantation and was shown to be sensitive to treatment by synthetic YFV 17D in this study.

### Example 6

### Treatment of Implanted Syngeneic CCL-53.1 Melanoma Cells in DBA 2 Mice with Low-Passage and High-Passage Synthetic YFV 17D

Female DBA/2 mice, aged 4-10 weeks, were acquired from Taconic Biosciences and bled for preliminary antibody titers on day −3. On day 0, mice from groups 3 and 5 were mock-vaccinated (see table 2). 8 mice based on minimum sample size calculations given the known standard deviation of tumor size from previous experiments (GraphPad StatMate). On day 21 and 35, vaccinated mice were bled and tested for neutralizing antibodies against YFV 17D using a plaque-reduction neutralization 50% (PRNT50) assay. On day 21, vaccinated mice were boosted with the same dose of the same virus as on day 0. Mice were implanted with 1×10⁵ CCL-53.1 cells in a volume of 100 μl DMEM through subcutaneous injection. All mice were treated as in Table 2 on days 51, 53, 56, 58, 60, 63, 65, 72, and 79 using a volume of 50 μl. Mice in groups 1, 2, 4, and 5 were treated an extra two times on days 88 and 93.

| | | | | | Sample |
|---|---|---|---|---|---|
| Group | Vaccination | Dose | Treatment | Dose (PFU) | Size |
| 3 | YFV 17D | 5 × 10⁶ | YFV 17D | 1 × 10⁷ | 8 |
| 5 | Mock | | Mock | | 8 |

Immunogenicity of YFV 17D: DBA/2 mice (n=8) were vaccinated on days 0 and 21, with sera collected on days 0, 21, and 35 for titration of neutralizing antibodies by PRNT50 assay. All mice were initially seronegative (GMT: <8) against YFV 17D, and after a single vaccination with 5×106 PFU all mice seroconverted (GMT: 172.3) on day 21. There was no significant difference in PRNT50 titers from day 21 to day 35 (GMT: 143.7) by paired t-test (p=0.3632). (See FIG. 6.)

Initial tumor size: Initial tumor sizes (day 51) for each group (n=8) were compared by ANOVA (p=0.3983) and Dunnett's multiple comparisons comparing each group with mock-vaccinated controls. The initial mean of tumors implanted YFV 17D vaccinated was smaller (37.18 mm³) compared to mock-treated (94.59 mm³) and this difference was significant by Student's t-test (p=0.020215) but not by ordinary one-way ANOVA or Dunnett's multiple comparisons test.

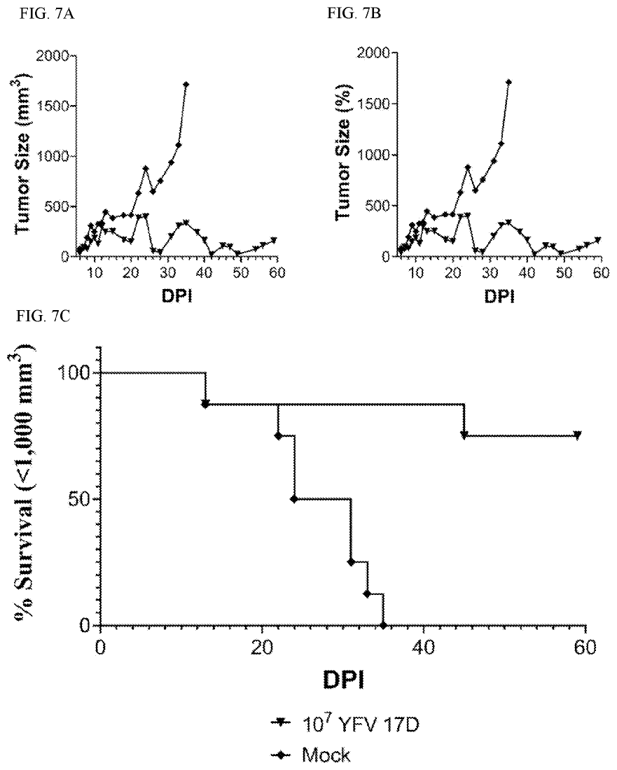

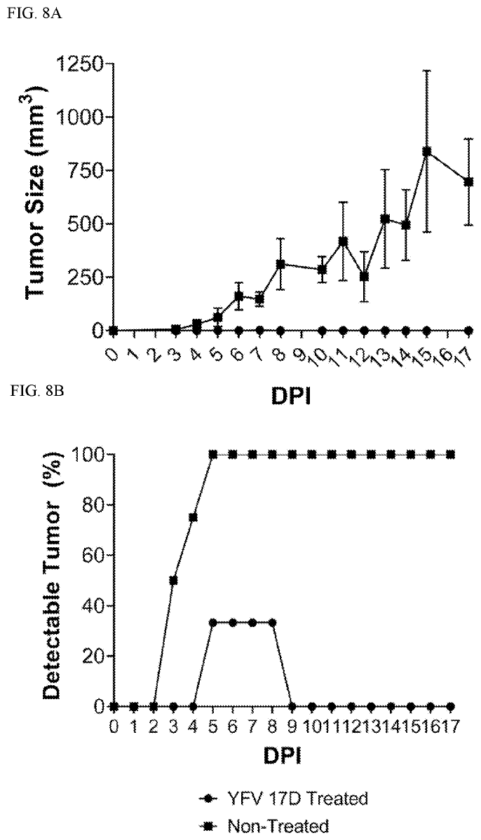

Efficacy of YFV 17D: Tumor sizes (mm³) were compared by multiple t-tests and found to be significantly smaller in YFV 17D treated mice on days 51, 53, 56, 63, 65, 69, 71, 73, 76, and 78. If you examine tumor growth as a function of percent change compared to the initial tumor size, there was no significant difference in YFV 17D treated versus mock-treated tumors at any day. However, survival (as determined by tumor size <1,000 mm³) was improved in YFV 17D treated tumors with a MTD of >60 compared to 27.5 in mock-treated tumors. (FIG. 7A-7C.)

A benefit was observed with each oncolytic treatment with improved survival and reduced tumor sizes for YFV 17D treatments. Survivors persisted from each treatment group with relatively low tumor sizes past 60 days post-implantation.

In conclusion, low-passage and high-passage YFV 17D are effective against melanoma using the syngeneic CCL-53.1 implantation model in DBA/2 mice.

### Example 7

### Successful Oncolytic Therapy with YFV 17D Prevents Further EMT-6 Tumor Growth Post-Challenge

BALB/C mice with YFV 17D treated and eradicated tumors (n=3) were challenged by implantation a second time with 10⁴ EMT6 TNBC cells. The mice were challenged by being injected subcutaneously into the right flank, a secondary site distant from the fat pad on the abdomen, the site of primary inoculation. Control, naïve, mice (n=8) were also implanted with 10⁴ EMT6 TNBC cells at the same time. The tumors in both groups were measured daily post-implantation. Tumor size (mm³) was significantly greater on days 4-14 post-implantation in the control mice. Although a small tumor appeared in a single mouse in the YFV 17D group on day 5, it disappeared on day 9. In the control group, tumors appeared in half the mice on day 3 and in all mice on day 5-14.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

1. A method of treating a malignant tumor or reducing tumor size, comprising:
administering attenuated Yellow Fever virus (YFV) to a subject in need thereof.

2. A method of treating a malignant tumor or reducing tumor size, comprising:
administering a prime dose of attenuated YFV to a subject in need thereof; and
administering one or more boost dose of attenuated YFV to the subject in need thereof.

3. (canceled)

4. The method of claim 1, wherein the attenuated YFV is YFV strain 17D vaccine (YFV 17D).

5. The method of claim 1, wherein the attenuated YFV is synthetic YFV strain 17D (YFV 17D).

6. The method of claim 1, wherein the attenuated YFV is YFV 17D-204, YFV 17DD, YFV 17D-213, codon deoptimized YFV, codon-pair deoptimized YFV, or YFV deoptimized by increasing CG or TA (or UA) dinucleotide content.

7. The method of claim 2, wherein the prime dose is administered subcutaneously, intramuscularly, intradermally, intranasally, or intravenously.

8. The method of claim 2, wherein the one or more boost dose is administered intratumorally or intravenously.

9. The method of claim 2, wherein a first of the one or more boost dose is administered about 2 weeks after one prime dose, or if more than one prime dose then about 2 weeks after the last prime dose.

10. The method of claim 2, wherein the subject has cancer.

11. The method of claim 2, wherein the prime dose is administered when the subject does not have cancer.

12. The method of claim 11, wherein the subject is at a higher risk of developing cancer.

13. The method of claim 11, wherein the one or more boost dose is administered about every 1, 2, 3, 4, 5, 6, 7, 8, 9 or 10 years after the prime dose when the subject does not have cancer.

14. The method of claim 11, wherein the subject is subsequently diagnosed with cancer and the one or more boost dose is administered after the subject is diagnosed with cancer.

15. The method of claim 1, wherein the method further comprises administering a PD-1 inhibitor or a PD-L1 inhibitor.

16. The method of claim 15, wherein the PD-1 inhibitor is an anti-PD1 antibody.

17. The method of claim 16, wherein the anti-PD1 antibody is selected from the group consisting of pembrolizumab, nivolumab, pidilizumab, AMP-224, AMP-514, spartalizumab, cemiplimab, AK105, BCD-100, BI 754091, JS001, LZM009, MGA012, Sym021, TSR-042, MGD013, AK104, XmAb20717, tislelizumab, and combinations thereof.

18. The method of claim 15, wherein the PD-1 inhibitor is selected from the group consisting of PF-06801591, anti-PD1 antibody expressing pluripotent killer T lymphocytes (PIK-PD-1), autologous anti-EGFRvIII 4SCAR-IgT cells, and combinations thereof and wherein the anti-PD-L1 inhibitor is M7824.

19. The method of claim 15, wherein the PD-L1 inhibitor is an anti-PD-L1 antibody.

20. The method of claim 19, wherein the anti-PD-L1 antibody is selected from the group consisting of BGB-A333, CK-301, FAZ053, KN035, MDX-1105, MSB2311, SHR-1316, atezolizumab, avelumab, durvalumab, BMS-936559, CK-301, and combinations thereof.

21. (canceled)

22. The method of claim 1,
wherein treating the malignant tumor decreases the likelihood of recurrence of the malignant tumor, or
wherein treating the malignant tumor decreases the likelihood of having a second cancer that is different from the malignant tumor, or
wherein if the subject develops a second cancer that is different from the malignant tumor, the treatment of the malignant tumor results in slowing the growth of the second cancer, or
wherein after remission of the malignant tumor, if the subject develops a second cancer that is different from the malignant tumor, the treatment of the malignant tumor results in slowing the growth of the second cancer.

23. (canceled)

24. (canceled)

25. (canceled)

26. The method of claim 1,
wherein treating the malignant tumor stimulates an inflammatory immune response in the tumor, or
wherein treating the malignant tumor recruits pro-inflammatory cells to the tumor, or
wherein treating the malignant tumor stimulates an anti-tumor immune response.

27. (canceled)

28. (canceled)

29. The method of claim 1, wherein the malignant tumor is a solid tumor.

30. The method of claim 1, wherein the malignant tumor is selected from a group consisting of glioma, neuroblastoma, glioblastoma multiforme, adenocarcinoma, medulloblastoma, mammary carcinoma, prostate carcinoma, colorectal carcinoma, hepatocellular carcinoma, bladder cancer, prostate cancer, lung carcinoma, bronchial carcinoma, epidermoid carcinoma, and melanoma.

31. The method of claim 1, wherein the attenuated YFV is administered intratumorally, intravenously, intracerebrally, intramuscularly, intraspinally or intrathecally.

32. The method of claim 31, wherein administering the attenuated YFV causes cell lysis in the tumor cells.
